(19) United States
(12) Patent Application Publication
(10) Pub. No.: US 2014/0342420 A1
(43) Pub. Date: Nov. 20, 2014

(54) PRODUCTION OF BIO-BUTANOL AND RELATED PRODUCTS

(71) Applicants: Guneet Kumar, Ellicot City, MD (US); Praveen Paripati, Reston, VA (US)

(72) Inventors: Guneet Kumar, Ellicot City, MD (US); Praveen Paripati, Reston, VA (US)

(73) Assignee: SUGANIT SYSTEMS, INC., Reston, VA (US)

(21) Appl. No.: 14/240,205

(22) PCT Filed: Aug. 22, 2012
(86) PCT No.: PCT/US2012/051908

§ 371 Date: Jun. 26, 2014

Related U.S. Application Data
(60) Provisional application No. 61/526,127, filed on Aug. 22, 2011.

(51) Int. Cl. C12P 7/16; C07C 67/08; C07C 29/147; C07C 67/317

(52) U.S. Cl. CPC C12P 7/16; C07C 67/317; C07C 67/08; C07C 29/147
USPC 435/160

(57) ABSTRACT

The present invention provides a method for the production of an alkanol from biomass. The method for producing an alkanol, including n-butanol, comprises fermenting a carbohydrate to produce a diacid and converting the diacid into an alkanol by solution chemistry or solid phase chemistry. The carbohydrate may be derived or extracted from a lignocellulose, an algae, a grain, or a starch containing root.

### CROSS-REFERENCE TO RELATED APPLICATIONS

This international patent application claims priority to U.S. Provisional Patent Application No. 61/526,127, filed Aug. 22, 2011, the disclosure of which is incorporated in its entirety.

### FIELD OF THE INVENTION

The present invention relates to the conversion of a diacid into an alcohol. More specifically, the invention relates to the conversion of succinic acid into n-butanol.

### BACKGROUND OF THE INVENTION

n-Butanol and ABE fermentation

Renewable n-butanol is a highly desirable product. It may be used as a drop-in replacement fossil fuel based gasoline. n-butanol may also be used as a starting material for various specialty chemical products such as butyl acrylate, butyl acetate, butylamines, and butenes—raw materials used in the manufacturing of plastics, rubber, fiber, polymers and other hydrocarbons such as, inter alia, jet fuel, biodiesel, or gasoline additives.

Considerable research has been done on the production of renewable n-butanol from the acetone-butanol-ethanol (ABE) fermentation process. The ABE fermentation process has been used in industrial processes since 1919. In this process, sugars, derived from various biomass sources, are fermented in the presence of a bacterial species of Clostridia. Ultimately, acetone, butanol, and ethanol are produced in a ratio of 3 parts acetone, 6 parts butanol, and 1 part ethanol.

Despite its routine practice, the ABE fermentation process presents significant drawbacks. In particular, the inability to scale-up the fermentation process due to product inhibition. The presence of butanol in and during the fermentation process inhibits the bacteria from producing additional quantities of butanol. This product inhibition leads to a lower yield of butanol with a final concentration of approximately 2% in the fermentation broth, making the process cost intensive.

To overcome this problem, several approaches have been developed. Among them include the development of a two stage fermentation processes and the development of genetically modified organisms that are more tolerant to the presence of the fermentation products and/or capable of reducing the production of fermentation by-products. Alternative approaches for butanol production also include fermentation and/or chemical modification of butanol isomers, such as 2-butanol.

### Succinic Acid

A C4 molecule similar to butanol that can also be produced from the fermentation process is succinic acid. Succinic acid fermentation has a few advantages over butanol fermentation. Microorganisms like Actinobacillus succinogenes can not only metabolize C6 sugars, but also C5 sugars generated from the cellulosic and hemicellulosic constituent of the renewable lignocellulosic feedstock. The literature reports that a maximum titer value of 14.5% succinic acid in the fermentation broth can be achieve before product inhibition sets in Okinawa, et al. (2008) Applied Microbiol. Biotechnology 81(3):459-464. This titer value is comparable to that of ethanol in the fermentation broth currently achieved by the fuel industry.

The fermentation process of succinic acid is also capable of producing higher yields of fermentation product per mole of sugar consumed. Succinic acid fermentation not only uses the carbon source from sugars, but is also capable of assimilating carbon dioxide. In fact, a report presented by the U.S. Department of Energy has identified succinic acid, a C4 diacid molecule, as one of the top value added chemicals because it can be produced in large quantities from the fermentation of biomass. Top Value Added Chemicals from Biomass, Volume 1: Results of Screening of Potential Candidates from Sugars and Synthesis Gas, August 2004, Office of Biomass Program.

Succinic acid can also be used to produce commercially valuable chemicals through well known hydrogenation or reduction reactions to yield Butanediol, Tetrahydrofuran (THF), and gamma-butyrolactone (GMB) family of compounds. GMB, for example, can be further reacted with amines to produce pyrrolidinones.

While the production of butanol from a variety of processes are known, there is a need to improve upon the limitations in the production methods currently available. The present invention provides an approach to the production of butanol, that avoids the fermentation inhibitor issue and at the same time increases the yield from a given sugar source by 50% to 100%. This approach significantly improves the production of renewable fuels and their viability.

### SUMMARY OF THE INVENTION

The present invention provides a method for the production of a 1-alkanol produced from biomass.

In one embodiment, a method for producing a 1-alkanol may comprise (a) fermenting a carbohydrate to produce a diacid; and (b) converting the diacid into 1-alkanol, optionally n-butanol. In another embodiment, the method further comprises purification of the diacid after fermentation. In another embodiment, the method may comprise converting the diacid by solution chemistry or solid phase chemistry.

In one embodiment, said carbohydrate may be derived or extracted from a lignocellulose, an algae, a grain or a starch containing root. In another embodiment, said carbohydrate may be a sugar. In another embodiment, said sugar may be a monosaccharide or a disaccharide. In another embodiment, said sugar may be glucose, fructose, sucrose, xylose, arabinose or any other sugar derived from a ligno-cellulose, an algae, a grain or a starch containing root.

In one embodiment, said fermenting may comprise fermenting in the presence of a microbe. In another embodiment, said microbe may be a fungus, a yeast, or a bacterium. In another embodiment, said microbe may be Aspergillus niger, Aspergillus fumigatus, Byssochlamys nivea, Lentinus degener, Paecilomyces varioti, Penicillium viniferum, mutants or recombinants thereof. In another embodiment, said microbe may be Saccharomyces cerevisiae, mutants or recombinants thereof. In another embodiment, said microbe may be Corynebacterium glutamicum, Enterococcus faecalis, Actinobaccillus succinogenes, recombinant Escherichia coli, Anaerobiospirillum succinicproduceus, Mannheimia succinicproduceus, mutants or recombinants thereof.

In one embodiment, said fermenting may be a single step fermentation process. In another embodiment, said fermenting may be a two step fermentation process. In another embodiment, said two step fermentation process may comprise the use of at least two different types of bacteria. In another embodiment, one of said at least two different types of bacteria comprise Rhizopus sp. or Enterococcus faecalis. In another embodiment, at least two different types of bacteria may comprise the combination of Rhizopus sp. and Enterococcus faecalis.

In one embodiment, said two step fermentation process comprises: (a) fermenting the carbohydrate to produce fumaric acid; and (b) fermenting said fumaric acid to produce succinic acid. In another embodiment, wherein said fermenting the carbohydrate may comprise fermentation with a Rhizopus sp. In another embodiment, comprising fermenting the fumaric acid may comprise fermentation with a Enterococcus faecalis.

In one embodiment, the method may further comprise converting the diacid into a cyclic anhydride. In another embodiment, the cyclic anhydride may form a 5-membered cyclic anhydride. In another embodiment, the cyclic anhydride may be a succinic anhydride or a butyrolactone. In another embodiment, the method may further comprise condensing the cyclic anhydride with an alcohol to form a mono ester. In another embodiment, the method may further comprise condensing the cyclic anhydride with an amine to form a mono amide. In another embodiment, the mono ester may be a 1-carboxy-4-ester. In another embodiment, the mono amide may be a 1-carboxy-4-amide. In another embodiment, the alcohol may be a primary alcohol. In another embodiment, the primary alcohol may be methanol, ethanol, propanol, pentanol, hexanol, or heptanol.

In one embodiment, the method may further comprise reducing in a first reduction reaction the mono ester to form a 1-hydroxy-4-ester. In another embodiment, the first reduction reaction may be in the presence of borane. In another embodiment, the method may further comprise reducing in a first reduction reaction the mono amide to form a 1-hydroxy-4-amide. In another embodiment, the first reduction reaction may be in the presence of borane. In another embodiment, the method may further comprise hydrolyzing the 1-hydroxy-4-ester to the corresponding 1-hydroxy-4-carboxylic acid and said 1-hydroxy-4-carboxylic acid further undergoing a second reduction to form a 1-alkanol. In another embodiment, the second reduction reaction may be by catalytic hydrogenation. In another embodiment, the method may further comprise hydrolyzing the 1-hydroxy-4-amide to the corresponding 1-hydroxy-4-carboxylic acid and said 1-hydroxy-4-carboxylic acid further undergoing a second reduction to form a 1-alkanol.

In one embodiment, the second reduction reaction may be in the presence of LiAlH₄ or LiBH₄. In another embodiment, the method may further comprise reducing the 1-hydroxy-4-ester to a 1-alkanol. In another embodiment, the method may further comprise reducing the 1-hydroxy-4-amide to a 1-alkanol.

In one embodiment, the method may further comprise the step of dehydrating the 1-alkanol to 1-alkene. In another embodiment, the method may further comprise the step of hydrating the alkene to 2-alkanol.

In one embodiment, the method for producing n-butanol may comprise fermenting a carbohydrate to produce a succinic acid and converting the succinic acid into n-butanol. In another embodiment, said succinic acid may be converted into n-butanol by solution chemistry or solid phase chemistry.

In another embodiment, said carbohydrate may be a sugar. In another embodiment, said sugar may be a monosaccharide or a disaccharide. In another embodiment, said carbohydrate may be derived or extracted from a ligno-cellulose, an algae, a grain or a starch containing root. In another embodiment, said sugar may be glucose, fructose, sucrose, xylose, arabinose or any other sugar derived from a ligno-cellulose, an algae, a grain or a starch containing root.

In one embodiment, said fermenting comprising fermenting in the presence of a microbe. In another embodiment, said microbe may be a fungus, a yeast, or a bacterium. In another embodiment, said microbe may be Aspergillus niger, Aspergillus fumigatus, Byssochlamys nivea, Lentinus degener, Paecilomyces varioti, Penicillium viniferum, mutants or recombinants thereof. In another embodiment, said microbe may be Saccharomyces cerevisiae, mutants or recombinants thereof. In another embodiment, said microbe may be Corynebacterium glutamicum, Enterococcus faecalis, Actinobaccillus succinogenes, recombinant Escherichia coli, Anaerobiospirillum succinicproduceus, Mannheimia succinicproducens, mutants or recombinants thereof.

In one embodiment, said fermenting may be a single step fermentation process. In another embodiment, said fermenting may be a two step fermentation process. In another embodiment, said two step fermentation process involves the use of at least two different types of bacteria. In another embodiment, one of said at least two different types of bacteria comprise Rhizopus sp. or Enterococcus faecalis. In another embodiment, at least two different types of bacteria may comprise the combination of Rhizopus sp. and Enterococcus faecalis. In another embodiment, said two step fermentation process may comprise fermenting the carbohydrate to produce fumaric acid; and fermenting said fumaric acid to produce succinic acid. In another embodiment, the method may comprise fermenting the carbohydrate to fumaric acid with a Rhizopus sp. In another embodiment, the method may comprise fermenting the fumaric acid to succinic acid with a Enterococcus faecalis.

In one embodiment, the method may further comprise converting the succinic acid into a succinic anhydride. In another embodiment, the method may further comprise condensing the succinic anhydride with an alcohol to form a mono ester. In another embodiment, the method may further comprise condensing the succinic anhydride with an amine to form a mono amide. In another embodiment, the mono ester may be a 1-carboxy-4-ester. In another embodiment, the mono amide may be a 1-carboxy-4-amide. In another embodiment, the alcohol may be a primary alcohol. In another embodiment, the primary alcohol may be methanol, ethanol, propanol, pentanol, hexanol, or heptanol.

In one embodiment, the method may further comprise reducing in a first reduction reaction the mono ester to form a 1-hydroxy-4-ester. In another embodiment, the first reduction reaction may be in the presence of borane. In another embodiment, the method may further comprise reducing in a first reduction reaction the mono amide to form a 1-hydroxy-4-amide. In another embodiment, the first reduction reaction may be in the presence of borane. In another embodiment, the method may further comprise hydrolyzing the 1-hydroxy-4-ester to the corresponding 1-hydroxy-4-carboxylic acid and said 1-hydroxy-4-carboxylic acid further undergoing a second reduction to form a 1-butanol. In another embodiment, the second reduction reaction may be by catalytic hydrogenation. In another embodiment, the method may further comprise hydrolyzing 1-hydroxy-4-amide to the corresponding 1-hydroxy-4-carboxylic acid and said 1-hydroxy-4-carboxylic acid further undergoing a second reduction to form a 1-butanol. In another embodiment, the second reduction reaction may be in the presence of LiAlH₄ or LiBH₄. In another embodiment, the method further comprise reducing the 1-hydroxy-4-ester to a 1-butanol. In another embodiment, the method further comprise reducing the 1-hydroxy-4-amide to a 1-butanol. In another embodiment, the method further comprise the step of dehydrating the 1-butanol to form an 1-butene. In another embodiment, the method may further comprise the step of dehydrating the 1-butene to 2-butanol.

In one embodiment, the method for producing 1-butanol may comprise (a) condensating succinic anhydride with an alcohol or amine to a mono ester or mono amide, respectively; and (b) reducing the mono ester or mono amide to said 1-butanol.

The present invention also provides a method for the production of a 1-alkanol produced from biomass. In one embodiment, the invention provides a method for the production of 1-alkanol from the fermentation of carbohydrates. In another embodiment, the carbohydrate fermentation process may produce a diacid, optionally succinic acid. In another embodiment, the diacid produced from the fermentation product may be produced without significant product inhibition, thereby producing an increased yield of diacids from a carbohydrate source, optionally biomass.

In another embodiment, the biomass may be agricultural residues, optionally corn stover, wheat straw, bagasse, rice hulls, or rice straw; wood and forest residues, optionally pine, poplar, douglas fir, oak, saw dust, paper/pulp waste, or wood fiber; algae; kudzu; coal; cellulose, lignin, herbaceous energy crops, optionally switchgrass, reed canary grass, or miscanthus; lingocellulosic biomass, optionally comprising lignin, cellulose, and hemicellulose; plant biomass; or mixtures thereof.

In another embodiment, the diacid produced from the fermentation process may be converted to a 1-alkanol. In another embodiment, 1-alkanol may be produced by converting the diacid through chemical reactions, thermal, physical, or electro-chemical means. In another embodiment, the 1-alkanol may be produced by converting the diacid by chemical means.

In another embodiment, the diacid may be first converted into a cyclic anhydride followed by a series of chemical reactions that produce the 1-alkanol.

In another embodiment, the diacid may be first attached to a solid support followed by a series of chemical reactions that produce the 1-alkanol. In another embodiment, the method first reduces the diacid to a diol and then attachs it to a solid support, followed by series of chemical reactions to ultimately produce the 1-alkanol. In another embodiment, the 1-alkanol produced may be n-butanol.

In a further embodiment, a method for producing n-butanol from biomass may comprise (a) converting biomass into carbohydrates, optionally a sugar; (b) converting said carbohydrate, optionally sugar, into a diacid, optionally succinic acid; and (c) converting said diacid, optionally succinic acid, to an alkanol, optionally 1-butanol.

In a further embodiment, a method for producing n-butanol from biomass may comprise (a) converting a carbohydrate, optionally sugar, into a diacid, optionally succinic acid; and (b) converting said diacid, optionally succinic acid, to an alkanol, optionally 1-butanol.

### DETAILED DESCRIPTION OF THE EMBODIMENTS

The present invention involves the production of a diacid, optionally succinic acid, by fermenting a carbohydrate, optionally a sugar, followed by the conversion of the diacid into a 1-alkanol, optionally butanol. This process occurs by a novel method using chemical, thermal, physical or electro-chemical means. Further, the carbohydrate may be derived from biomass. Methods for producing sugars from biomass are known in the art. See U.S. Pat. No. 8,030,030.

This method comprises converting a symmetrical diacid molecule produced by a fermentation process to an asymmetrical alkanol, optionally butanol. As an example, this conversion is achieved by selectively reacting one of the carboxylic groups of a succinic acid to render the molecule asymmetric. Selective chemical reactions are performed on the two reactive moieties of the asymmetical molecule to ultimately yield butanol.

The combination of the fermentation processes to yield succinic acid followed by a chemical, thermal, physical or electro-chemical means to produce a 1-alkanol is a highly attractive approach. For example, in the case of producing n-butanol, (a) both succinic acid and butanol are molecules with four carbon atoms, which allows for the transformation of a molecule of succinic acid to a molecule of butanol through one or more steps; (b) succinic acid can be fermented from carbohydrates derived from ligno-cellulosic biomass, algal biomass, grains or roots containing starch i.e., this process consumes both carbon dioxide and the carbons from the carbohydrate sugars to produce a higher yield of succinic acid; and (c) high concentrations of succinic acid in the fermentation broth makes the process less capital intensive.

The combination of the two processes results in substantial increase in the production of bio-butanols. The bio-butanol produced by this method can be subsequently reacted, by for example dehydration and/or hydration, to yield other products such as but not limited to butene and/or 2-butanol.

### A. Fermentation of Carbohydrates.

Carbohydrates useful in the present invention are those that are derived from and/or extracted from biomass sources. These may include, inter alia, carbohydrates from ligno-cellulose, algae, grain, or starch containing roots. The carbohydrates useful in the present invention may include monosaccharides and disaccharides, which are generally referred to as sugars. Sugars that may be used in the fermentation process include, inter alia, glucose, fructose, sucrose, xylose, arabinose, or any other sugar derived from ligno-cellulose, algae, grain, or starch containing roots. Further, sugars may be derived from biomass.

The fermentation of various carbohydrates is well known in the art. For example, the fermentation of succinic acid is reviewed by Song, et al. (2006) Enzyme and Microbial Technology 39:352-361. Generally, a carbohydrate, such as those described herein, are fermented in the presence of a microbe. The microbe has the ability, through its own biochemical machinery, to convert the carbohydrate or sugar into useful by-products such as succinic acid.

As can be appreciated from the foregoing, microbes that may be used in the present invention may include those capable of fermenting carbohydrates or sugars into diacid by-products. These microbes may include fungus, yeast, or bacteria. Fungus useful for this invention may include, for example, Aspergillus niger, Aspergillus fumigatus, Byssochlamys nivea, Lentinus degener, Paecilomyces varioti, Penicillium viniferum, mutants or recombinants thereof. Yeast useful for this invention may include, for example, Saccharomyces cerevisiae, mutants or recombinants thereof. Bacteria useful for this invention may include, for example, Corynebacterium glutamicum, Enterococcus faecalis, Actinobaccillus succinogenes, recombinant Escherichia coli, Anaerobiospirillum succinicproduceus, Mannheimia succinicproducens, mutants or recombinants thereof. The microbe for the production of succinic acid is Actinobaccillus succinogenes, Anaerobiospirillum succinicproduceus, or Mannheimia succinicproducens.

The fermentation process for the production of succinic acid may occur via a single step or by at least two steps. Under a single step fermentation process, bacteria such as Actinobaccillus succinogenes will metabolize a sugar directly into succinic acid as the major fermentation by-product. In a two step fermentation process, two different sets of bacteria are utilized. The first bacteria is used to produce an intermediate by-product, while the second bacteria is utilized to finalize the intermediate by-product into the final by-product. For example, in the production of succinic acid, glucose and rice bran are fermented into fumaric acid by Rhizopus species. Subsequently, the fumaric acid is fermented into succinic acid by Enterococcus faecalis. Those of skill in the art will appreciate which fermentation process and the number of steps required to most effectively produce the diacid by-product used in the production of the alkanol.

Prior to the conversion of the diacid into the alkanol, the fermentation broth comprising the diacid may also be filtered or manipulated to extract the desired by-product. For example, in the fermentation of a sugar to succinic acid, the fermentation broth comprising the desired succinic acid may be filtered or extracted such that the succinic acid is purified from the broth. Subsequently, the purified succinic acid may be converted to the desired end product of n-butanol.

B. Conversion of the Diacid into Alkanol

Once the desired diacid is extracted or purified from the fermentation broth, if such a step is required, a series of chemical, thermal, physical, or electro-chemical reactions or means may be utilized to convert the diacid into the desired alkanol. The diacid may be chemically converted to form the alkanol. Such a chemical reaction may be accomplished by either solution chemistry, solid phase chemistry or a combination of solution and solid phase chemistry.

By utilizing a chemical reaction, for example, the chemistry involves masking or protecting one of the carboxylic acid groups of the diacid. The remaining carboxylic acid group that is not masked or not protected is selectively reduced. After the unmasked carboxylic acid moiety is reduced, the masked acid group is deprotected and subsequently reduced to an 1-alkanol. The diacid utilized may be succinic acid and the 1-alkanol may be n-butanol.

(1) Solution Chemistry

To synthesize an asymmetric 1-alkanol from a diacid, the diacid is heated to form a cyclic anhydride. The cyclic anhydride formed maybe a 5 or 6 membered ring. In one embodiment, the cyclic anhydride formed is a 5-membered ring. In one embodiment, the cyclic anhydride may be a succinic anhydride or a butyrolactone.

The highly reactive cyclic anhydride intermediate allows for selective reactivity with either an alcohol or amine to form a mono ester or a mono amide, respectively. In one embodiment, the mono ester is a 1-carboxy-4-ester. In another embodiment, the mono amide is a 1-carboxy-4-amide. The reaction of the alcohol or amine with the cyclic anhydride may also take place by any other condensation reaction known by those of skill in the art. The alcohols used in the condensation reaction may be any type of alcohol, most preferred, the alcohol is a primary alcohol. In one embodiment, the primary alcohol is a methanol, ethanol, propanol, pentanol, hexanol, or heptanol.

Following the condensation reaction, the mono ester or mono amide undergoes a first reaction in which the unprotected carboxy group is reduced to a hydroxy group. Thus in one embodiment, the resulting product of the first reduction reaction is either a 1-hydroxy-4-ester or a 1-hydroxy-4-amide. The reducing agent used in this first reduction may include, for example, borane. Alternatively, the carboxylic group maybe converted to an acid halide and subsequently reduced to the alcohol. The reducing agent used for this first reduction may include, for example, NaBH₄, DIBAL, catalytic hydrogenation or electrochemical reduction.

The hydroxyl moiety of the resulting 1-hydroxy-4-ester or 1-hydroxy-4-amide of the first reduction reaction is subsequently converted to an active leaving group (for example sulfonate ester or halide). The resulting modified 1-hydroxy-4-ester or 1-hydroxy-4-amide is then subject to a second reduction reaction in the presence of a reducing agent to form the 1-alkanol. The reducing agent used may include for example, LiAlH₄ LiBH₄, or catalytic hydrogenation for the ester, and LiAlH₄ or LiBH₄ for the amide.

(2) Solid Phase Chemistry

The chemistry outlined above can also be applied to a solid surface by using a membrane, bead or any other solid surface that has an alcohol or amine attached. The alcohol or amine reacts directly with a diacid (C2 to C7) to form an immobilized amide or ester. In this method, there is no need to generate the cyclic anhydride to form the asymmetric molecule. Reduction reactions as described above can be performed on the asymmetric molecule immobilized to the solid support. Once reduced, the molecule can be released from the solid support and further reduced to a 1-alkanol simultaneously. In one embodiment, the diacid used in the solid phase chemistry is a succinic acid and the 1-alkanol is a n-butanol.

(3) Solution and Solid Phase Combination Chemistry

The chemistry outlined above can also be applied as a combination of solution and solid phase chemistry where the diacid is first reduced by hydrogenation, reduction or electrochemical means to a diol. Then the diol is reacted with a carboxylic group which is attached to a solid support (membrane, bead, or any other solid phase configuration) to yield an ester. Chemistry as described above can be done on this asymmetric molecule to produce 1-alkanol. However, after the last reduction step the solid support has an alcohol moiety instead of a carboxylic group. This alcohol group can be oxidized to regenerate/recycle the solid support. In one embodiment, the diacid used in this combination chemistry is succinic acid which is reduce to butanediol using solution chemistry and then further reduced to 1-butanol using solid phase chemistry.

(4) Other Reactions

The 1-alkanol products produced by either one of the chemistries, outlined above, can be applied in a further reaction to produce other products. For example, if n-butanol is produced using the chemistries outlined above, the n-butanol may undergo a dehydration reaction to form 1-butene. Subsequently, 1-butene may further undergo an additional hydration reaction to form 2-butanol.

All publications (e.g., Non-Patent Literature), patents, patent application publications, and patent applications mentioned in this specification are indicative of the level of skill of those skilled in the art to which this invention pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

Although methods and materials similar or equivalent to those described herein may be used in the invention or testing of the present invention, suitable methods and materials are described herein. The materials, methods and examples are illustrative only, and are not intended to be limiting.

The invention now being generally described, it will be more readily understood by reference to the following examples, which are included merely for purposes of illustration of certain aspects and embodiments of the present invention, and are not intended to limit the invention.

### EXAMPLES

### Example 1

### Glucose to n-butanol

Glucose is fermented in the presence of Actinobaccillus succinogens to produce succinic acid. The succinic acid is purified from the fermentation broth. The purified succinic acid, hydroxymethyl polystyrene and catalytic p-toluenesulfonic acid are heated to reflux (105° C.) in toluene for 21 hours in an apparatus equipped with a Dean-Stark trap for dehydration of the reaction mixture. The free carboxy moiety of the immobilized mono succinate ester is subsequently reduced in the presence of BH₃ in THF (0° C. to 25° C. for 18 hours) to form the immobilized 1-hydroxy-4-ester. The resulting immobilized 1-hydroxy-4-ester is converted to the immobilized 1-methanesulfonyloxy-4-ester by reaction with methane sulfonyl chloride and pyridine in THF at ambient temperature for 2 hours. Finally, the immobilized 1-methanesulfonyloxy-4-ester is reduced in the presence of LiBH₄ (THF, 0° C. to 25° C. over 2 hours) to release n-butanol.

### Example 2

### Succinic Acid to Jet Fuel

Succinic acid oligomers, specifically dimers, trimers and tetramers can be reduced to hydrocarbons to produce jet fuel and diesel. Gamma rays and UV rays can be used to produce above oligomers from succinic acid.

Oligomeric succinic acid can be reduced using reducing agents to the oligomeric alchol, which can be subsequently dehydrated to give the oligomeric-ene molecule. These oligomericene molecules can be further hydrogenated to give a mixture of C8, C12, C16 and higher level hydrocarbons for jet fuel.

Those skilled in the art will recognize, or be able to ascertain using no more than routine experimentation, many equivalents to the specific embodiments of the invention described herein. Such equivalents are intended to be encompassed by the following claims.

1. A method for producing a 1-alkanol comprising
(a) fermenting a carbohydrate to produce a diacid; and
(b) converting the diacid into 1-alkanol.

2-3. (canceled)

4. The method of claim 1, wherein said carbohydrate is derived or extracted from a lignocellulose, an algae, a grain or a starch containing root.

5-7. (canceled)

8. The method of claim 1, wherein said fermenting comprises fermenting in the presence of a microbe.

9-11. (canceled)

12. The method of claim 8, wherein said microbe is Corynebacterium glutamicum, Enter ococcus faecalis, Actinobaccillus succinogenes, recombinant Escherichia coli, Anaerobiospirillum succinicproduceus, Mannheimia succinicproducens, mutants or recombinants thereof.

13. (canceled)

14. The method of claim 1, wherein said fermenting is a two step fermentation process.

15-17. (canceled)

18. The method of claim 14, wherein said two step fermentation process comprises:
(a) fermenting the carbohydrate to produce fumaric acid; and
(b) fermenting said fumaric acid to produce succinic acid.

19-20. (canceled)

21. The method of claim 1, further comprising converting the diacid into a cyclic anhydride.

22-23. (canceled)

24. The method of claim 21, further comprising condensing the cyclic anhydride with an alcohol to form a mono ester.

25. The method of claim 21, further comprising condensing the cyclic anhydride with an amine to form a mono amide.

26-29. (canceled)

30. The method of claim 24, further comprising reducing in a first reduction reaction the mono ester to form a 1-hydroxy-4-ester.

31. (canceled)

32. The method of claim 25, further comprising reducing in a first reduction reaction the mono amide to form a 1-hydroxy-4-amide.

33. (canceled)

34. The method of claim 30, further comprising hydrolyzing the 1-hydroxy-4-ester to the corresponding 1-hydroxy-4-carboxylic acid and said 1-hydroxy-4-carboxylic acid further undergoing a second reduction to form a 1-alkanol.

35. (canceled)

36. The method of claim 32, further comprising hydrolyzing the 1-hydroxy-4-amide to the corresponding 1-hydroxy-4-carboxylic acid and said 1-hydroxy-4-carboxylic acid further undergoing a second reduction to form a 1-alkanol.

37-41. (canceled)

42. A method for producing n-butanol comprising fermenting a carbohydrate to produce a succinic acid and converting the succinic acid into n-butanol.

43-47. (canceled)

48. The method of claim 42, wherein said fermenting comprising fermenting in the presence of a microbe.

49-51. (canceled)

52. The method of claim 48, wherein said microbe is Corynebacterium glutamicum, Enterococcus faecalis, Actinobaccillus succinogenes, recombinant Escherichia coli, Anaerobiospirillum succinicproduceus, Mannheimia succinicproducens, mutants or recombinants thereof.

53. (canceled)

54. The method of claim 42, wherein said fermenting is a two step fermentation process.

55-60. (canceled)

61. The method of claim 42, further comprising converting the succinic acid into a succinic anhydride.

62. The method of claim 61, further comprising condensing the succinic anhydride with an alcohol to form a mono ester.

63. The method of claim 61, further comprising condensing the succinic anhydride with an amine to form a mono amide.

64-67. (canceled)

68. The method of claim 62, further comprising reducing in a first reduction reaction the mono ester to form a 1-hydroxy-4-ester.

69. (canceled)

70. The method of claim 63, further comprising reducing in a first reduction reaction the mono amide to form a 1-hydroxy-4-amide.

71. (canceled)

72. The method of claim 68, further comprising hydrolyzing the 1-hydroxy-4-ester to the corresponding 1-hydroxy-4-carboxylic acid and said 1-hydroxy-4-carboxylic acid further undergoing a second reduction to form a 1-butanol.

73. (canceled)

74. The method of claim 70, further comprising hydrolyzing 1-hydroxy-4-amide to the corresponding 1-hydroxy-4-carboxylic acid and said 1-hydroxy-4-carboxylic acid further undergoing a second reduction to form a 1-butanol.

75-77. (canceled)

78. The method of claim 42, further comprising the step of dehydrating the 1-butanol to form an 1-butene.

79. The method of claim 78, further comprising the step of dehydrating the 1-butene to 2-butanol.

80. (canceled)

81. A method for producing n-butanol from biomass comprising
(a) converting biomass into carbohydrates, optionally sugars;
(b) converting said carbohydrate, optionally sugar, into a diacid, optionally succinic acid; and
(c) converting said diacid, optionally succinic acid, to an alkanol, optionally 1-butanol.

82. (canceled)
